(19) United States
(12) Patent Application Publication
(10) Pub. No.: US 2010/0211089 A1
(43) Pub. Date: Aug. 19, 2010

(54) Cutting Device for a Curved Corneal Channel

(75) Inventors: Mark Tomalla, Mulheim a. d. Ruhr (DE); Gebhard Tomalla, Essen (DE)

Correspondence Address: Sunstein Kann Murphy & Timbers LLP, 125 SUMMER STREET, BOSTON, MA, 02110-1618, US

(73) Assignees: Karin Tomalla, Mulheim a.d. Ruhr (DE); Jutta Tomolla, Essen (DE); ACRI. TEC GMBH, Hennigsdorf (DE)

(21) Appl. No.: 12/670,293

(22) PCT Filed: Jul. 28, 2008
(86) PCT No.: PCT/EP08/06217

§ 371 Date: Apr. 15, 2010

(30) Foreign Application Priority Data
Jul. 26, 2007 (DE) 10 2007 035 447.0

(51) Int. Cl. A61F 9/007

(52) U.S. Cl. 606/166

(57) ABSTRACT

Device for cutting a curved channel into the cornea (105) of an eye, with a holding means (301, 302) that can be applied to the cornea (105) in a radial direction defined by the radius of curvature of the cornea (105), and with a cutting means (303) that has a curved blade (303.3) for cutting the curved channel, wherein the holding means (301, 302) has a guide means (301.2) with which the blade (303.3) for cutting the channel is guided in a plane extending transversely with respect to the radial direction, and wherein the holding means (301, 302) is designed to define the position of the blade (303.3) in the radial direction during the cutting of the channel.

If defective vision of the eye is caused by malformation of the cornea, then it is possible to correct the defect by implanting support rings into the cornea. In order to be able to carry out an implantation of this type, channels have to be cut into the cornea as a receptacle for these rings, usually there are two ring halves. This takes place for example with the aid of femtosecond lasers, a very precisely operating method, or in a purely manually mechanical manner, for example with the aid of a blade shaped as a semicircular ring. For this purpose, a radially running cut, reaching down to a specific depth, is first made into the cornea. Subsequently, the tip of the blade is inserted into a perpendicular cut face.

An annular blade of this type is for example known from Keravision U.S. Pat. No. 5,403,335.

The circular blade, which extends over approximately 180° and runs horizontally, is connected to a hollow cylindrically embodied handle which is arranged perpendicularly thereto and can be used to operate the blade unit by hand.

The application of the tilted blade, the resetting to the ideal position, the holding-down and horizontally guiding while at the same time rotationally pivoting the blade about a virtual central axis are in themselves requirements which already harbour imprecisions in execution and above all the high risk of piercing of the cornea.

The object of the invention is to provide a device which eliminates the above-mentioned drawbacks and risks while maintaining known blade units.

Starting from a device according to the preamble of claim 1, the present invention achieves this object by the features specified in the characterising part of claim 1. Furthermore, starting from a method according to the preamble of claim 18, it achieves this object by the features specified in the characterising part of claim 18.

The present invention is based on the technical teaching that a particularly beneficial and precise configuration of the corneal channels is achieved if, during the cutting of the respective channel, the blade is not only guided transversely to its axis of rotation, but also experiences axial securing, thus allowing a precise cut to be carried out.

In preferred variants of the invention, the construction of the device is broken down primarily into two halves which, viewed from above, have the area of a semicircle (half-plates).

The connection between the cornea and the device, which advantageously is formed in the contact region e.g. as a 35 dpt arched spherical cap, is produced preferably by evacuating.

The spherical cap can have on its surface two special features which are each formed on a specific radius. These may be, on the one hand, an evacuable depression formed in the spherical cap as a rounded groove and, on the other hand, a thin-walled trough likewise running in a semicircle as a displacer or hold-down means.

The presence of both special features is optional, but not absolutely essential. It is for example also possible that just one half is equipped with the groove and that there is no trough, or that there is a trough but no groove. The latter variant ensures that the blade can be inserted completely free from transverse forces into the cornea and guided further therein.

The device makes it possible to receive blade units for cutting circularly running channels into the cornea, which are modified only slightly and pertain to the prior art. The blade unit is mounted in the device in a centred manner so as to be rotatable and vertically adjustable.

As a result of the two radial cuts, which may be necessary, in the cornea, it is expedient not to advance the channel starting from one radial cut up to the second but to cut it only up to shortly therebefore, owing to the fixation of the support ring to be inserted.

Subsequently, the blade is pivoted back as a result of a counter-rotational movement at the handle and the channel is rinsed.

If an annular channel is also to be cut into the remaining half of the cornea, then the device has to be offset through 180°, as a result of which the half of the cornea that is already equipped with the channel is held down and the other half is raised if appropriate.

If, in this case too, the channel was formed, the cutting blade removed and the channel rinsed, then the support ring can subsequently be inserted into this half. This process is conceivable, even if only one support ring is to be implanted, before the device is removed from the cornea.

The eventual presence of two radial incisions into the cornea eliminates the need to use two contrarily designed blades, such as are necessary with one radial cut, namely one for the cut in the clockwise direction and a second for the counter-clockwise cut.

Further preferred embodiments of the invention will be apparent from the dependent claims or the subsequent description of preferred exemplary embodiments that refers to the appended drawings. It is shown in:

FIG. 1 a schematic view of a first part of a preferred embodiment of the device according to the invention;

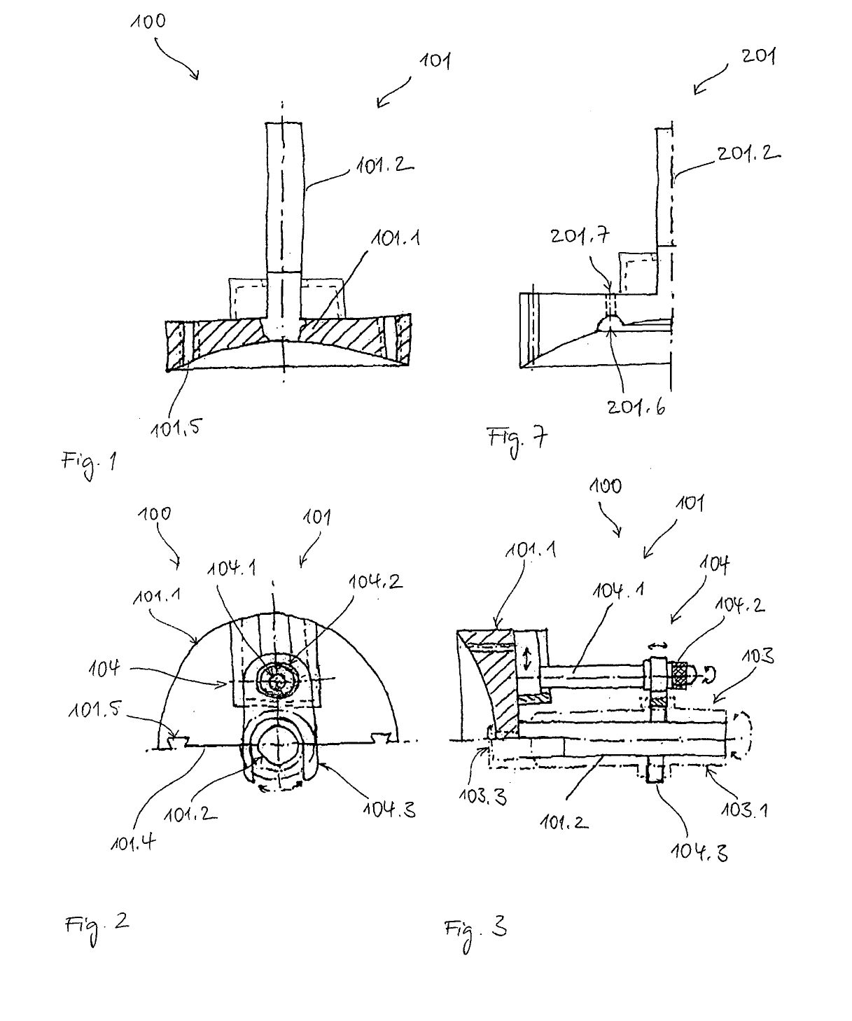

FIG. 2 a schematic plan view onto the part of FIG. 1;

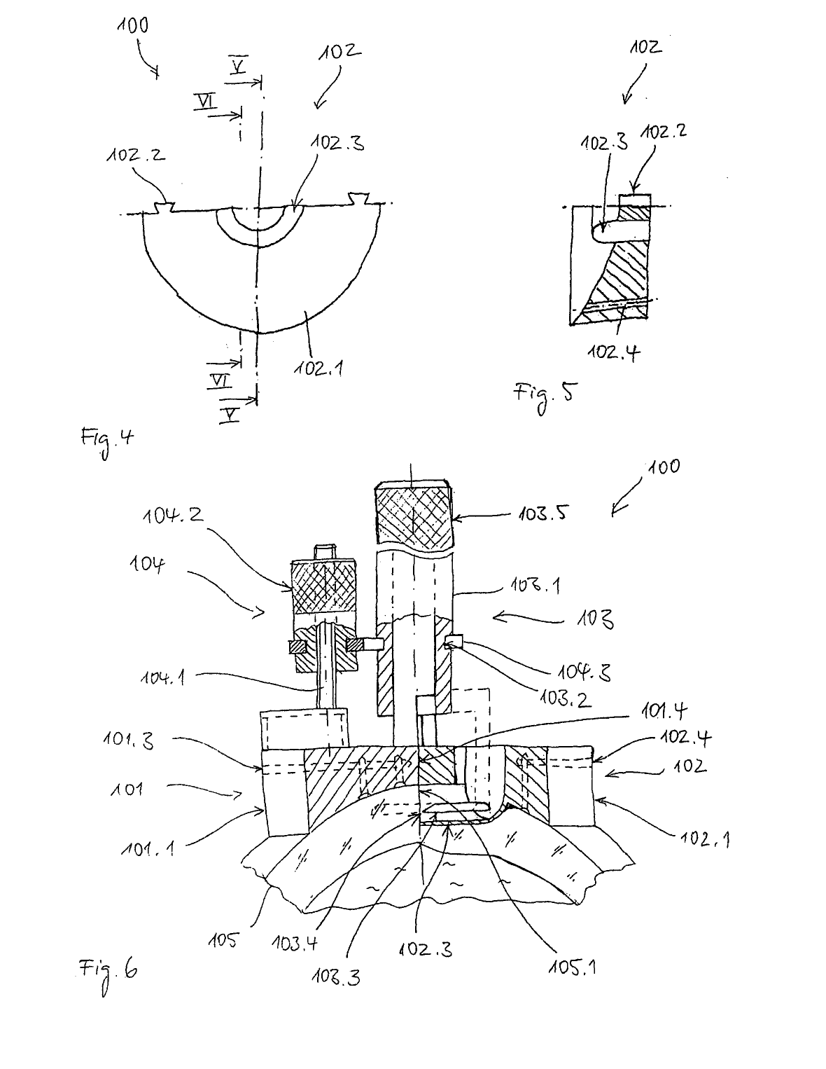

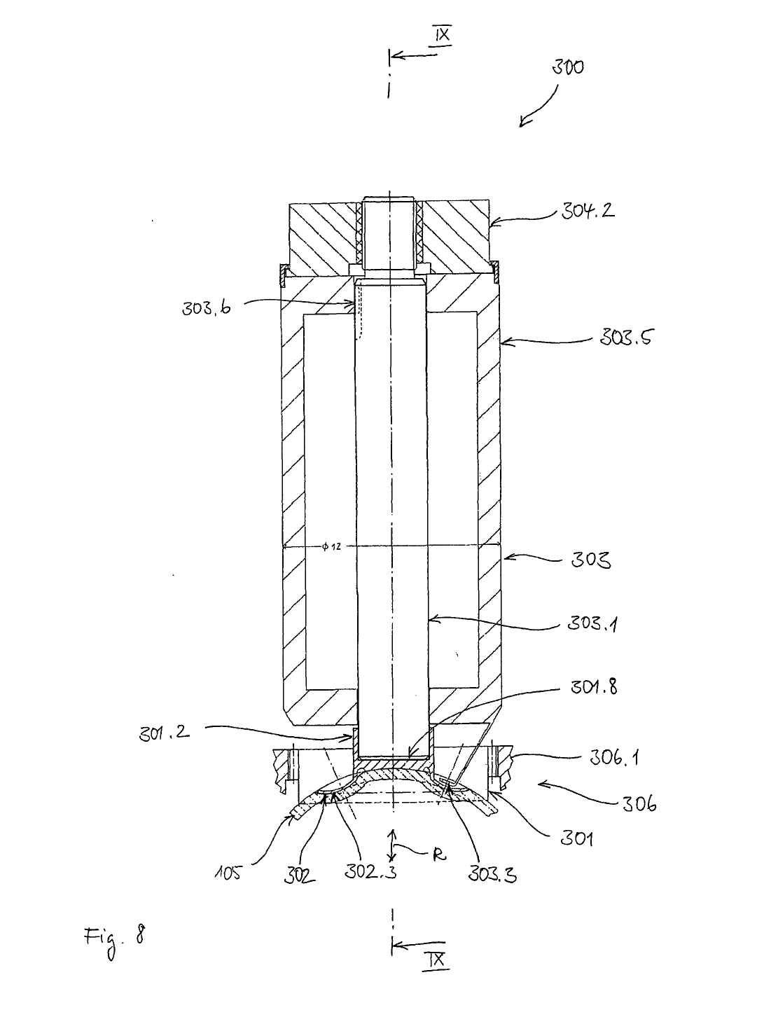

FIG. 3 a schematic partial section through the part of FIG. 2 along the line III-III;

FIG. 4 a schematic plan view onto a second part of the device of FIG. 1;

FIG. 5 a schematic partial section through the second part of FIG. 4 along the line V-V;

FIG. 6 a schematic section through the device according to the invention of FIGS. 1 and 4 along the line VI-VI in the assembled state;

FIG. 7 a schematic view of a first part of a further preferred embodiment of the device according to the invention;

FIG. 8 a schematic view of a further preferred embodiment of the device according to the invention;

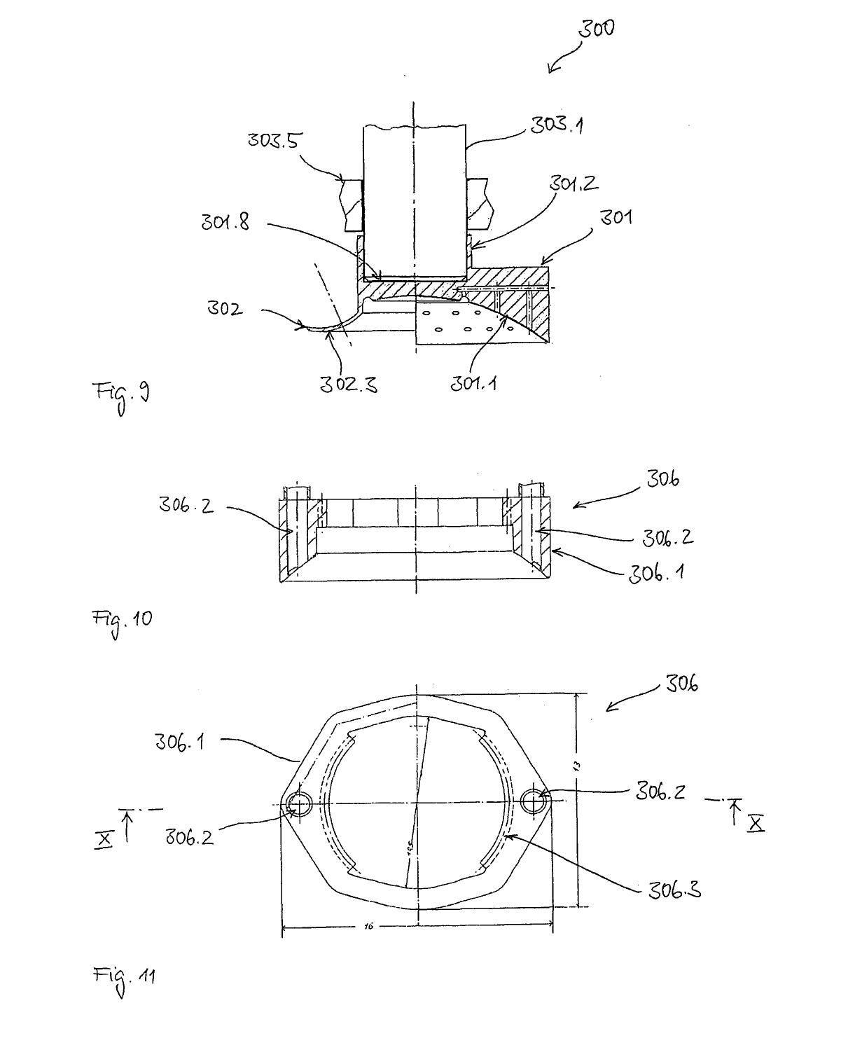

FIG. 9 a schematic sectional view of a part of the device (along the line IX-IX) of FIG. 8;

FIG. 10 a schematic sectional view of a fixing ring of the device of FIG. 8;

FIG. 11 a schematic plan view onto the fixing ring of FIG. 10;

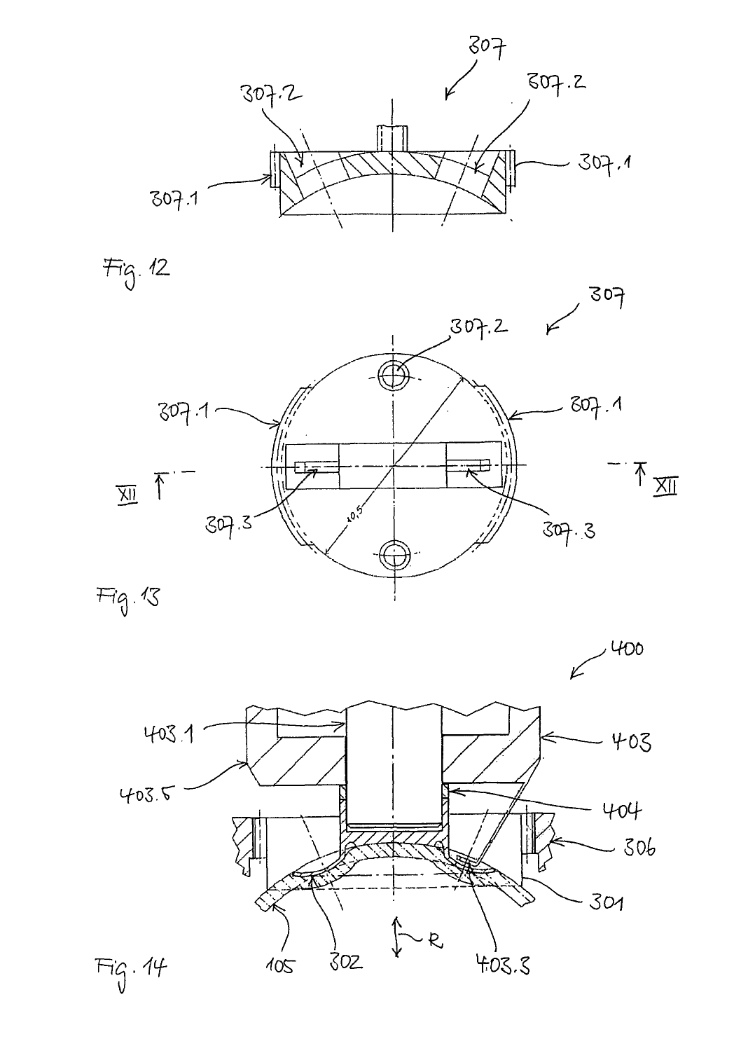

FIG. 12 a schematic sectional view of a cutting template for the device of FIG. 8;

FIG. 13 a schematic plan view onto the cutting template of FIG. 12; and

FIG. 14 a schematic sectional view of a part of a further preferred embodiment of the device according to the invention.

### FIRST EMBODIMENT

As a first embodiment, a variant in which the holding means of the device 100 has a half 101 which is embodied as a simple spherical cap, i.e. without a groove, and a second half 102 which is designed with a trough will be described hereinafter with reference to FIGS. 1 to 6. Both parts 101 and 102 each have a semicircular rest 101.1, 102.1. The part 101 is equipped with a central shaft 101.2 serving as a bearing means on which a hollow cylinder 103.1 of a cutting means in the form of a blade unit 103 is rotatably or pivotably mounted in a manner allowing height adjustment.

To allow setting and adjustment of the height of the blade unit 103 along the radial direction (defined by the radius of curvature of the cornea 105), on the base side, a height setting unit 104 comprising a fixable micrometre screw 104.1 is eccentrically slid or pivoted onto the part 101 in a form-fitting manner. A knurled nut 104.2, which forms a height adjustable region, is positioned on the micrometre screw 104.1. A fork 104.3 is rotatably mounted on the knurled nut 104.2 and engages the annular groove 103.2 located on the hollow cylinder 103.1 in a precise fit and thus brings the blade unit 103 into the setpoint height position and holds it there.

The part 101, without the blade unit 103 and height setting unit 104, is aligned and positioned on the cornea 105 and is fastened thereto by drawing a vacuum (via suitable channels 101.3). Subsequently, a cut depth-adjustable blade, (not shown), which is guided at the separating edge 101.4 of the part 101, is used to form two incisions in the cornea 105 in a manner matched to the diameter of the channel or support ring, respectively. In other words, the part 101 thus forms a cutting template for these incisions.

In the next step, vertical longitudinal guides 101.5, 102.2, for example, are used to connect the part 102 to the part 101 and to press the trough 102.3—flush with the radial incisions—into the cornea 105 sufficiently far until the two spherical cap halves 101.1, 102.1 of the part 101 and the part 102 are at the same height. Subsequently, the spherical cap 102.1 of the part 102 is securely connected to the cornea 105, likewise by evacuating (via suitable channels 102.4).

In other words, the trough 102.3 therefore forms a hold-down unit which is arranged in the region of the blade movement space of the blade 103.3 that is passed through during cutting of the channel and displaces, from the blade movement space, the part of the cornea 105 that is not to be provided with the channel during the current cut.

The blade unit 103, which is equipped with rolling and/or sliding bearings, is now slid onto the central shaft 101.2 of the part 101. Here, the blade unit 103 is to be positioned in such a way that the blade 103.3 having the shape of an arc of a circle fits into the arc of a circle of the trough 102.3 in an appropriate manner.

The height of the blade 103.3 is adjusted using the above-described height setting unit 104. Subsequently thereto, the blade tip 103.4 is inserted into the end wall 105.1, which is exposed as a result of the pressing-down of the trough-side half of the cornea 105, by means of rotary or pivoting movement manually triggered at the handle 103.5 of the blade unit 103, and the channel is cut at approximately 180° of the circumference. The blade 103.3 is returned by reversing the pivoting direction, the height adjustment means 104 is detached from the part 101 and the blade unit 103, and the blade unit 103 is removed from the central shaft 101.2. The part 102 is ventilated and raised from the cornea 105 and the part 101. The part 101 is ventilated and also removed from the cornea 105, or is placed onto the half of the cornea 105 that is equipped with the (previously cut) channel, respectively, in a manner rotated through 180°, to be precisely flush with the radial cuts, and the assembly and also (after the cutting the second channel) the disassembly are carried out in the known manner.

After the two channels cut have been rinsed, the respective support rings can be implanted.

### SECOND EMBODIMENT

A further preferred embodiment of the device 200 according to the invention will be described hereinafter with reference to FIG. 7. This device 200, in its design and mode of operation, basically corresponds to the device 100 so that, to avoid repetitions, reference is made to the foregoing discussion and merely the differences will be outlined. In particular, like components are provided with reference numerals increased by a value of 100. With regard to the features of these components, unless otherwise stated hereinafter, reference is expressly made to the foregoing discussion.

FIG. 7 (in a half view corresponding to the view from FIG. 1) shows a variant of the device 200 according to the invention using a first part 201 which is slightly modified compared to the first part 101.

The difference with respect to the variant described hereinbefore consists in the fact that, after the radial cuts have been made, the cornea 105 is not pressed down by the trough 102.3 on the side that is currently not to be provided with the channel in order to expose the end wall 105.1 of the cornea 105 for applying the blade 103.3. Instead, the side of the cornea 105 is drawn into the groove 201.6 (i.e. upwards in FIG. 7) via a groove 201.6 adapted to the curvature of the blade 103 and suction channels 201.7 connected to said groove in order to raise the end wall 105.1 of the cornea 105 and thus to expose it for applying the blade 103.3.

In other words, the groove 201.6 and the suction channels 201.7 are therefore part of a lifting unit which is arranged in the region of the blade movement space of the blade 103.3 being passed through during cutting of the channel and which lifts the part of the cornea 105 to be provided with the channel during the current cut into the blade movement space.

Subsequently, as in the first variant, the blade 103.3 is then positioned via the height adjustment means 104 (at the height of the groove 201.6) and applied to the end wall 105.1 (positioned in the groove 201.6 at that time) and the channel is cut as described above.

In this instance, provision may be made to cover the open ends of the groove with a cover (for example a film or the like) in order to ensure that the cornea 105 is drawn sufficiently into the groove. The cover is then pierced during cutting of the channel.

It goes without saying that the second part 102 is not necessarily required in this variant. In particular, there does not have to be a trough. Nevertheless, an annular second part can be helpful for supporting the device against a tilting of the central shaft 201.2 (serving to guide the blade unit 103).

### THIRD EMBODIMENT

A further preferred embodiment of the device 300 according to the invention will be described hereinafter with reference to FIGS. 8 to 13. This device 200, in its design and mode of operation, basically corresponds to the device 100 so that, to avoid repetitions, reference is made to the foregoing discussion and merely the differences will be outlined. In particular, like components are provided with reference numerals increased by a value of 200. With regard to the features of these components, unless otherwise stated hereinafter, reference is expressly made to the foregoing discussion.

In this variant, the holding means of the device 100 comprises a half 301 which is designed as a simple calotte, i.e. without a groove, whereas the second half 302 is designed, again, with a trough-like hold-down means. Both parts 301 and 302 are connected to each other in one piece and each form a semicircular rest 301.1, 302.1.

The component 301, 302 is equipped with a bearing bush 301.2 which serves as a bearing means and in which a shaft means, in the form of a pin 303.1 of a cutting means in the form of a blade unit 303, is rotatably or pivotably mounted. The distal end of the pin 303.1 facing the cornea 105 is positioned on a stop face 301.8 of the bearing bush 301.2, so that the position of the pin 303.1 in the radial direction R is defined as a result.

The blade unit 303 comprises a handle 303.5 to which the blade 303.3 is fastened. The handle 303.5 is connected to the pin 303.1 in a rotationally fixed but longitudinally displaceable manner, again, via a rotation prevention means 303.6.

For setting and adjustment of the height of the blade unit 303 along the radial direction R (defined by the radius of curvature of the cornea 105), a height setting unit 104 is provided on the handle 303.5 at the end facing away from the cornea 105. The height setting unit 104 comprises a threaded wheel (or a knurled nut, respectively) 304.2 which is connected to the handle 303.5 in a manner to be rotatable but secured in the axial direction, and which is screwed to an end-side thread of the pin 303.1. Via this threaded wheel 304.2 the blade unit 303, in the radial direction R, can be brought into the desired setpoint height position (above the cornea 105) and can be held there.

If appropriate, it is possible to provide a means for arresting the threaded wheel 104.2 preventing maladjustment of the position once it is set. However, if appropriate, an arresting means of this type may also be dispensed with, since maladjustment during actuation of the blade unit 303 via the handle 303.5 is prevented via the rotation prevention means 303.6.

The component 301, 302 is held via a fixing ring 306.1 (illustrated in greater detail in FIGS. 10 and 11) of a fixing means 306. First, the fixing ring 306.1 is applied to the cornea 105 and aligned. Subsequently, a reduced pressure, via which the fixing ring 306.1 is fixed on the cornea 105, is generated via suction channels 306.2.

The fixing ring 306.1, on its inner side, has a toothing 306.3, for example a spur toothing, or other fixing means which can be used to connect the component 301, 302 to the fixing ring 306.1 in a rotationally fixed manner (by means of suitable counterpieces).

After the fixing ring 306.1 has been aligned and positioned on the cornea 105 and fastened thereto by drawing a vacuum, a cutting template 307 (not shown in greater detail in FIGS. 12 and 13) is firstly inserted into the fixing ring 306.1.

The cutting template 307 comprises at its outer circumference a toothing 307.1 which interacts with the toothing 306.3 of the fixing ring 306.1 and therefore connects the cutting template 307 to the fixing ring 306.1 in a rotationally fixed manner.

Subsequently, a reduced pressure, via which the cutting template 307 is fixed on the cornea 105, is generated via suction channels 307.2.

Subsequently, a cut depth-adjustable blade (not shown), which is guided through openings 307.3 in the cutting template 307, is used to form two incisions in the cornea 105 in a manner matched to the diameter of the channel or support ring, respectively.

In the next step, the cutting template 307 is removed from the fixing ring 306.1 and the component 301, 302 is inserted into the fixing ring 306.1. This takes place in such a way that the trough 302.3—in a manner flush with the previously formed radial cuts—is pressed into the cornea 105 until the calotte half 301.1 rests against the cornea 105. Subsequently, the component 301, 302 is securely connected to the cornea 105 by evacuation (via suitable channels 301.3 and 302.4).

In other words, the trough 302.3 therefore forms a hold-down unit which is arranged in the region of the blade movement space of the blade 303.3 passed through during cutting of the channel and which displaces the part of the cornea 105 not to be provided with the channel during the current cut from the blade movement space.

Now, the blade unit 303 is inserted with the pin 303.1 into the bearing bush 301.2 (which may, if appropriate, also be provided with corresponding rolling and/or sliding bearings). In this instance, the blade unit 303 is to be positioned in such a way that the arc of a circle shaped blade 103.3 enters the arc of a circle of the trough 302.3 in an appropriate manner.

The height of the blade 303.3 is adjusted using the above-described height setting unit 304. Subsequently thereto, the blade tip 303.4 is inserted into the end wall 105.1, which is exposed as a result of the pressing-down of the trough-side half of the cornea 105, by means of rotary or pivoting movement manually triggered at the handle 303.5 of the blade unit 303, and the channel is cut at approximately 180° of the circumference.

The blade 103.3 returned by reversing the pivoting direction, the blade unit 303 is detached. The component 301, 302 is ventilated and raised from the fixing ring 306.1 and the cornea 105, if appropriate, rotated through 180° and placed flush with the radial cuts back onto the cornea 105, and the assembly and also (after the cutting of the second channel) the disassembly are carried out the a known manner.

The fixing ring can be provided with an appropriate scale or marking to facilitate application of the component 301, 302. This marking can, if appropriate, also be used as early as when the radial cuts are made by means of the cutting template 307, and therefore facilitates the positioning of the components flush with the radial cuts.

After the two cut channels have been rinsed, the respective support rings can be implanted.

### FOURTH EMBODIMENT

A further preferred embodiment of the device 400 according to the invention will be described hereinafter with reference to FIG. 14. This device 400, in its design and mode of operation, basically corresponds to the device 300 so that, to avoid repetitions, reference is made to the foregoing discussion and merely the differences will be outlined. In particular, like components are provided with reference numerals increased by a value of 100. With regard to the features of these components, unless otherwise stated hereinafter, reference is expressly made to the foregoing discussion.

FIG. 14 shows (in a partial view corresponding to the view from FIG. 8) a variant of the device 400 according to the invention in which the height setting unit is implemented via exchangeable spacer rings 404 via which the height of the blade 303.3 can be set in the radial direction R.

The blade unit 403 therefore differs from the blade unit 303 merely to the extent that the pin 403.1 can be rigidly connected to the handle 403.5.

What is claimed is:

1-21. (canceled)

22. A device for cutting a curved channel into the cornea of an eye with
a holding means applicable to the cornea in a radial direction defined by the radius of curvature of the cornea, and
a cutting means having a curved blade for cutting the curved channel,
the holding means comprising a guide means guiding the blade for cutting the channel in a plane running transversely to the radial direction,
characterised in that
the holding means is adapted to define the position of the blade in the radial direction during cutting of the channel.

23. A device according to claim 22, characterised in that
the holding means comprises a hold-down unit,
the hold-down unit being arranged in the region of the blade movement space of the blade passed through during cutting of the channel and
the hold-down unit being adapted to displace a part of the cornea not to be provided with the channel during the current cut from the blade movement space.

24. A device according to claim 23, characterised in that the hold-down unit comprises a portion of trough-shaped design for receiving the blade.

25. A device according to claim 22, characterised in that
the holding means comprises a lifting unit,
the lifting unit being arranged in the region of the blade movement space of the blade passed through during cutting of the channel and
the lifting unit being adapted to lift a part of the cornea to be provided with the channel during the current cut into the blade movement space.

26. A device according to claim 25, characterised in that the lifting unit comprises a portion of trough-shaped design for receiving the blade.

27. A device according to claim 25, characterised in that the lifting unit comprises a suction means for drawing the part of the cornea to be provided with the channel during the current cut into the portion of trough-shaped design.

28. A device according to claim 22, characterised in that a height setting unit is provided by means of which the position of the blade with respect to the cornea is adjustable along the radial direction.

29. A device according to claim 28, characterised in that the height setting unit comprises a screw means adjusting the position of the blade with respect to the cornea along the radial direction.

30. A device according to claim 28, characterised in that the height setting unit comprises an exchangeable spacer element setting the position of the blade with respect to the cornea along the radial direction.

31. An instrument set for cutting a curved channel into the cornea of an eye with
a device according to claim 1 and
a cutting template for forming, in the cornea, at least one incision pointing in the radial direction,
the holding means having a fixing means for selectively fixing the guide means and the cutting template to the cornea.
